(19) United States
(12) Patent Application Publication
(10) Pub. No.: US 2006/0134012 A1
(43) Pub. Date: Jun. 22, 2006

(54) Temporary pharmacologically-inactive dental coating for the in situ protection of dental therapeutic agents from saliva and abrasion from chewing

(76) Inventors: John M Symington, Etobicoke (CA); Brenda Turgeon, Oakville (CA); Oliver Ross Perry, Toronto (CA)

Correspondence Address: Benita J Rohm;Rohm & Monsanto, 12 Rathbone Place, Grosse Pointe, MI, 48230, US

(21) Appl. No.: 10/539,923

(22) PCT Filed: Dec. 16, 2003
(86) PCT No.: PCT/CA03/01943

§ 371 Date: Feb. 21, 2006

Related U.S. Application Data
(60) Provisional application No. 60/433,933, filed on Dec. 16, 2002.

(51) Int. Cl. A61K 8/81

(52) U.S. Cl. 424/49

(57) ABSTRACT

Topical application of a pharmacologically-inactive, inert polymeric solution to teeth affords a temporary, protective barrier for pharmacologically-active compounds that have been previously applied topically to the tooth surfaces for caries reduction. In a method of use, an aqueous dispersion polymethylmethacrylate (PMMA) and a suitable plasticizer are applied topically to the tooth surface immediately after the topical application of chlorhexidine or fluoride. The inert coating thus formed is distinct from any pharmacologically-active substance, and remains on the tooth surface until abraded by the chewing of hard foods. In a composition of matter, the solution contains ammonio methacrylate copolymer, type B USP/NF of between 20% and 35% w/w; triethyl citrate USP/NF of between 1% and 10% w/w; and purified water of between 60% and 70% w/w. The ammonio methacrylate copolymer is preferably EUDRAGIT RS 30 D brand available form Rohm GmbH.

### RELATIONSHIP TO OTHER APPLICATIONS

This application claims the benefit of U.S. Provisional Patent Application No. 60/433,933 filed Dec. 16, 2002.

### BACKGROUND OF TH INVENTION

1. Field of the Invention

The invention relates to a composition of a pharmacologically-inactive coating that acts as a temporary mechanical barrier to protect an underlying layer containing pharmacologically-active agents, such as dental therapeutic agents, residing on a tooth surface, against erosion from salivary washings and abrasion from eating foods, and methods of use thereof.

2. Description of the Related Art

Dental caries is a chronic, asymptomatic, transmittable bacterial infection on the surface of the teeth, which affects about 20% of the population. It is the most prevalent chronic disease in adults affecting about one in three adults over the age of 50, and is also the most common pediatric disease. The dental research literature and common dental practice support the use of two common dental therapeutic compounds to combat dental caries, namely: the antimicrobial compound chlorhexidine, and the remineralizing compound fluoride. Both compounds may be administered separately in varnish modalities of varying concentrations by the dental professional to the teeth of patients in the dental office. Varnishes containing these therapeutic agents have the benefit of delivering the agent to the site of caries at relatively high concentrations compared to oral rinses or gels, of delivering these agents for reportedly long periods of time in the oral cavity, and ensuring patient compliance.

In Europe and Canada where they are approved for dental use by regulatory bodies, chlorhexidine varnishes are an accepted standard of preventive dental care.

Commercially available chlorhexidine-containing varnishes include those sold under the trademarks PREVORA (10% (w/v) chlorhexidine) by CHX Technologies, Inc., Toronto, Canada; EC40 (up to 40% (w/w) chlorhexidine) by Certichem, Nijmegen, Netherlands; and CERVITEC (1% wt chlorhexidine) by Ivoclar Vivadent, Liechtenstein.

It is reported in the scientific literature that the varnish modality is able to deliver chlorhexidine in vitro for between 24 hours and 3 months. See, for example, S. Matthijs and P. Adriaens, “Chlorhexidine Varnishes: A Review,” Journal of Clinical Periodontology, Vol. 29, pp. 1-8 (2000). One particular varnish involves two separate and sequentially applied coatings, the first being a pharmacologically active solution containing chlorhexidine, and the second being a coating of commercially-available dental polyurethane with methylene chloride as the solvent. See, H. J. Sandham, et al., “A Preliminary Report of Long-term Elimination of Detectable Mutans Streptococci in Man,” J. Dent. Res., Vol. 67, No. 1, pp. 9-14 (1988); H. J. Sandham, et al., “Clinical Trial in Adults of an Antimicrobial Varnish for Reducing Mutans Streptococci,” J. Dent. Res., Vol. 70, No. 11, pp. 1401-1408 (1991); and H. J. Sandham, et al., “The Effect of Chlorhexidine Varnish Treatment on Salivary Mutans Streptococcal Levels in Child Orthodontic Patients,” J. Dent. Res., Vol. 71, No. 1, pp. 32-35 (1992).

This two-stage dental varnish used by Sandham, et al. is described in U.S. Pat. No. 4,883,534 issued Nov. 28, 1989. In particular, this patent describes the second stage coating as polymeric liquid film of polyurethanes or polylacetates which cures in situ to form a hard transparent or translucent film over the chlorhexidine varnish that further retards release of the active ingredient from the first stage coating. The patent described the resident time for this second coating, which preferably also contains a therapeutic agent, such as fluoride, as at least four days.

Banting, et al. reported significant reductions of caries in a controlled clinical study of high-risk adult patients using a two-stage chlorhexidine varnish (see, D. Banting, et al., “The Effectiveness of 10% Chlorhexidine Varnish Treatment on Dental Caries Incidence in Adults with Dry Mouth,” Gerodontology, Vol. 17, No. 2, pp. 67-76 (2001)). The second stage of this varnish was a separate solution, which was applied directly over the chlorhexidine coating, and consisted of 29% (w/w) polyurethane, 22% ethyl acetate and 49% acetone.

Inert coatings for sustained drug delivery of pharmaceutical tablets in the gastrointestinal tract have been extensively described in the scientific literature and in the patent literature, and have been widely used by the pharmaceutical industry. Of particular interest to this invention is the EUDRAGIT brand family of polymethylmethacrylates (PMMAs) marketed by Rohm Pharma Polymers of Rohm GmbH, Darmstadt, Germany. A description of the EUDRAGIT brand polymers can be found, inter alia, on the internet at http://www.rohmamerica.com/Eudragit/HomePage.html. EUDRAGIT brand polymers have also been used as excipients in sustained release dosage forms and to form transdermal drug delivery systems.

The use of EUDRAGIT brand, or other PMMA-type polymers, to deliver drugs in the oral cavity, has been described in the patent literature as follows:

U.S. Pat. No. 5,160,737 issued Nov. 3, 1992, titled “Liquid Polymer Composition, and Method of Use” describes a liquid methacrylic acid copolymer, including EUDRAGIT acrylics, containing a releasing agent and a pharmacological agent for sustained drug release in the oral cavity.

U.S. Pat. No. 5,330,746 issued Jul. 19, 1994 titled “Dental Varnish Composition and Method of Use” describes an oral composition for plaque prevention and tooth hypersensitivity consisting of an antibacterial agent embedded in a carrier such as an acrylic polymer, and preferably an EUDRAGIT acrylic, and the use of this composition to prevent caries.

U.S. Pat. No. 6,197,331, Mar. 6, 2001, titled “Pharmaceutical Oral Patch for Controlled Release of Pharmaceutical Agents in the Oral Cavity” describes a EUDRAGIT-based device containing active pharmaceutical compounds in a polymer matrix which is not bonded to the teeth and which can be removed from the mouth.

U.S. Patent Application No. 20010024657 published Sep. 27, 2001 titled “Pharmaceutical Oral Patch for Controlled Release of Pharmaceutical Agents in the Oral Cavity,” which is a continuation-in-part to U.S. Pat. No. 6,197,331, describes a singular oral composition possibly containing PMMA, including EUDRAGIT polymers, and a pharmaceutically active component for application to the teeth.

References in the scientific literature to methacrylic coatings for drug release purposes in the oral cavity include the following:

Diarra, et al. describe a 200 mg tablet consisting of a granular matrix of hydroxyapatite, ethyl cellulose and EUDRAGIT polymers, along with an active drug substance, which is then fixed on to the tooth for sustained release of the pharmacologically active substance; see, M. Diarra, et al., “Elaboration and Evaluation of an Intraoral Controlled Release Delivery System,” Biomaterials, Vol. 19, pp. 1523-1527 (1998).

Patel, et al. describe a rigid polymeric device consisting of polyethyl methacrylate and tetrahydrofurfuryl plus chlorhexidine for the reduction of fungus in the oral cavity; the device was tested in vitro for its reduction of Candida albicans. See M. Patel, et al., “A polymeric system for the intra-oral delivery of an anti-fungal agent,” Biomaterials, Vol. 22, pp. 2319-2324 (2001).

The aforementioned patents and scientific publications incorporating PMMA-type polymers in the oral cavity are directed to one-stage drug delivery formulations, tablets or devices which combine the acrylic polymer, nominally from the EUDRAGIT family of acrylics, with an active drug substance. As will be discussed further hereinbelow, this architecture differs significantly from the present invention in which the pharmacologically-active substance is applied to the surfaces of teeth as a first coating, or layer, that is separate from an inert, inactive polymer coating that forms a temporary mechanical barrier in situ against erosion of the first layer.

It is also noted that PMMA has been used widely in the oral cavity as evident from its approval for use in the U.S. Code of Federal Regulations (CFR) Chapter 21, and in particular Section 872.3820 for root canal filling resins, Section 872.3770 for crown and bridge resins, Section 872.3760 for denture relining, repairing and rebasing resin, Section 872.3750 for bracket adhesive resin and tooth conditioner, and Section 827.3765 for pit and fissure sealant and conditioner. In all these uses, however, PMMA is applied on permanent basis and/or as part of an oral device of rigid structure.

The use of triethyl citrate as an acceptable plasticizer for EUDRAGIT methacrylic polymers is published in Rohm's technical literature. Triethyl citrate is also an approved plasticizer in the U.S. Code of Federal Regulations Chapter 21, Section 181.27.

However, in no instance does Rohm's technical or marketing literature describe the direct application of EUDRAGIT polymers to the surfaces of teeth as an inert, pharmacologically inactive coating in and of itself; nor does the aforementioned patent or scientific literature. In the context of drug delivery in the oral cavity, the EUDRAGIT brand polymers are considered as an excipients or additives in a pharmacologically-active composition or device.

### SUMMARY OF THE INVENTION

In a first aspect of the invention, a formulation for a pharmacologically-inactive, inert polymer coating comprising a PMMA-type polymer and a plasticizer is provided as a liquid that can be applied as a film to the surface of a tooth in the oral cavity of a dental patient. The inert polymer coating, when placed on top of a temporary coating of a pharmacologically-active substance applied to a surface of the tooth, functions as a temporary mechanical barrier to delay erosion of the active substance due to salivary washing and abrasion from the eating of food, and thereby enhances retention of the pharmacologically-active agents that have been applied as a coating to tooth surfaces.

Such pharmacologically-active agents include, without limitation, the antimicrobial compound chlorhexidine, and the remineralizing compound fluoride, both of which are known to be effective in the treatment and prevention of dental caries.

Clorhexidine, for example, has a bitter taste, and the provision of an inert mechanical barrier layer helps to improve the acceptance of these therapeutic agents by the patient and, hence, the patient's compliance to treatment. Thus, higher concentrations of therapeutic agents can be administered to the patient over a longer period of time.

Because the formulation is intended for as an oral composition, it should have acceptable taste, clarity, color, durability and application characteristics for the dental professional, as well as biocompatibility to permit its use on patients' teeth as a separate temporary protective coating for dental therapeutic compounds.

In a preferred embodiment of this aspect of the invention, an aqueous dispersion of a polymethylmethacrylate copolymer includes a sufficient amount of a plasticizer to form a film in situ when applied to a tooth surface that has appropriate flexibility and that dries quickly in the oral cavity. Polymethylmethacrylate copolymers, suitable for the practice of the invention, are commercially available, such as the copolymers of methacrylic acid and methacrylate marketed by Rohm GmbH, Darmstadt, Germany as the EUDRAGIT brand family of polymethylmethacrylates.

In a particularly preferred embodiment of the invention, the polymethylmethacrylate is an ammonio methacrylate copolymer type B USP/NF, such as EUDRAGIT RS 30 D brand polymethylmethacrylate. EUDRAGIT RS 30 D conforms to the specifications of an ammonio methacrylate copolymer, Type B in the U.S. Pharmacopeia and the U.S. National Formulary. EUDRAGIT RS 30D, which is an aqueous dispersion of acrylic polymer, has been used according to the manufacturer's directions of use and industry standards, as an approved pharmaceutical and cosmetic excipient in oral and dermal applications for drug delivery in solid dosage forms in the gastrointestinal tract or on the skin for many years.

Suitable plasticizers for the PMMA include pharmaceutical grade triethyl citrate, dibutyl sebacate, dibutyl phthalate, and diethyl phthalate, and the like. Triethyl citrate, however, is particularly preferred. Preferred concentrations of plasticizer range from between 1% w/w and 20% w/w. In the formulation process, the plasticizer is preferably added to the aqueous dispersion of PMMA over a period of between 10 and 30 minutes.

The liquid formulation of the present invention, preferably, has a viscosity of between 5 cP and 30 cP, and is preferably between 5 cP and 20 cP, and a specific gravity of 1.054 g/ml plus or minus 0.050 g/ml.

In a preferred embodiment of the invention, the formulation comprises a liquid dispersion (w/w) of:

20% to 35% ammonio methacrylate copolymer type B USP/NF;

1% to 10% triethyl citrate; and

60% to 70% purified water.

In a particularly preferred specific embodiment of the invention, the formulation comprises (w/w):

28% EUDRAGIT RS 30 D polymethylmethacrylate;

6% triethyl citrate; and

66% w/w water.

In a method of use aspect of the invention, a formulation in accordance with the present invention is applied to the surfaces of teeth to form a pharmacologically inert barrier coating over prior coating(s) containing pharmacologically-active substances.

Preferably, the pharmacologically-active substance(s) comprise one or more active agents of the type known to reduce caries when applied to the tooth, illustratively, chlorhexidine and/or fluoride.

The liquid formulation which is clear, tasteless, and odorless, can be applied by a dental professional with a brush or cotton balls, or by any other means, so as to form, when dried, a film in situ on the tooth surface. The formulation dries within seconds, preferably after using an air syringe. Once formed, the inert barrier coating formed by the composition of the invention remains intact on the surface of the teeth for as long as the patient avoids chewing hard foods, such as meat, raw fruit, or crusty rolls. The coating is not affected by chewing soft foods such as soup and cheese. Nor is the coating affected by heat, such as by drinking hot beverages.

As used herein the singular term tooth, includes plural teeth, in other words, the inert barrier coating may be applied to any and all tooth surfaces in the mouth of a patient. The amount of inert barrier coating applied will typically range from about between 200 μL and 600 μL depending on the size of mouth and number of teeth of the patient.

In a further method aspect of the invention, a method of preventing or reducing the incidence of caries in teeth, includes the steps of applying a liquid coating of pharmacologically-active substances of the type used to reduce caries to a tooth surface; followed by applying a pharmacologically inert barrier coating, in accordance with the present invention, on top of the coating containing the pharmacologically-active substance. The inert barrier coating serves as a temporary mechanical barrier to delay erosion of the therapeutic coating caused by the washings of saliva and abrasion caused by eating food. Preferably, the coatings are dried by the dental professional.

Of course, the components used in the practice of the method aspect of the invention can be provided as a two component kit, the ingredients of which are capable, of reducing caries in the oral cavity. This embodiment would provide a separate compositions, the first composition including a therapeutic agent, such as chlorhexidine and/or fluoride and the second composition in accordance with the present invention comprising, for example, a PMMA in an aqueous dispersion with sufficient triethyl citrate to ensure flexibility and rapid drying in the oral cavity.

### DETAILED DESCRIPTION OF THE INVENTION

In accordance with a specific illustrative embodiment of the invention, a pharmacologically inert, bio-acceptable, liquid formulation of a water-suspended PMMA and plasticizer is formulated as follows:
28% EUDRAGIT RS 30 D polymethylmethacrylate;
6% triethyl citrate; and
66% w/w water.

A dental professional applies this formulation to the surface of teeth in a dental patient with a brush following the application of a coating of a pharmacologically-active substance, specifically chlorhexidine and/or fluoride in accordance with methods that are known and practiced in the art. Preferably, the first coating is dried, such as with an air syringe, or is permitted to dry prior to application of the pharmacologically-inert barrier coating.

In the clinical results reported herein, the pharmacologically-active layer was a chlorhexidine (10% w/v) solution sold under the trademark PREVORA by CHX Technologies, Inc., Toronto, Canada.

Clinical Results

Durability Testing:

The liquid formulation was evaluated in vivo for durability and patient acceptability on six different occasions separated by 48 hours or more, by one volunteer human subject. The formulation once colored with red food dye for ease of observation, was self-applied by this volunteer to the buccal (outer) surfaces of the upper central incisor teeth plus the upper two canine teeth. The volunteer visibly observed the durability of the coating over the course of both day and night, and during meals.

Recorded observations showed that the patient found this coating to be acceptable to taste and feel by the tongue as well as durable for a period of more than 12 hours or the period when the volunteer did not eat. The coating was abraded from the tooth surface by chewing hard foods, but not by chewing soft foods.

Protection of Pharmacologically-Active Substances on the Tooth Surface:

A pharmacologically-active liquid coating of chlorhexidine (10% w/v) was applied by a dental professional to the buccal (other) upper right central incisor teeth and the upper canine tooth of six volunteer subjects. The chlorhexidine coating was then air dried. Immediately thereafter, an aqueous dispersion of the formulation set forth in this section was applied over top the first coating of chlorhexidine and was also air dried.

At various intervals over 24 hours, the buccal surfaces of these teeth were tested for chlorhexidine residue by way of dry blotter paper discs placed on the teeth surfaces for one minute. The presence or absence of chlorhexidine on these paper discs was then tested by way of standard microbiology procedures involving zones of inhibition, using a strain of Streptococcus mutans bacteria in agar. The test was conducted over a 24 hour period and involved discs retrieved from the tooth surface at 1 hour, 2 hours, 4 hours and 24 hours after application of the inert coating herein described. In 3 of 6 patients for a period of up to 2 hours after application of the liquid coating, the discs inhibited bacterial growth and hence contained chlorhexidine. In the other 3 of 6 patients, the discs provided no inhibition to bacterial growth at any time. This clinical experiment illustrated that the PMMA coating prevented contact between the paper disk and the chlorhexidine under-layer at most points of observation up to 24 hours after application.

Although the invention has been described in terms of specific embodiments and applications, persons skilled in the art may, in light of this teaching, generate additional embodiments without exceeding the scope or departing from the spirit of the claimed invention. Accordingly, it is to be understood that the drawing and description in this disclosure are proffered to facilitate comprehension of the invention and should not be construed to limit the scope thereof.

What is claimed is:

1. A formulation for a pharmacologically inert coating to serve as a temporary mechanical barrier on top of a temporary coating of a pharmacologically-active substance applied to a surface of a tooth, the formulation comprising:
an aqueous dispersion of a polymethylmethacrylate; and
a plasticizer.

2. The formulation of claim 1 wherein the polymethylmethacrylate is an ammonio methacrylate copolymer, type B USP/NF.

3. The formulation of claim 2 wherein the ammonio methacrylate copolymer is EUDRAGIT RS 30 D brand polymethylmethacrylate.

4. The formulation of claim 1 wherein the plasticizer is a pharmaceutical grade plasticizer selected from the group consisting of triethyl citrate, dibutyl sebacate, dibutyl phthalate, and diethyl phthalate.

5. The formulation of claim 4 wherein the plasticizer is triethyl citrate.

6. The formulation of claim 4 comprising between 1% and 20% w/w plasticizer.

7. The formulation of claim 6 having a viscosity of between 5 cP and 30 cP and a specific gravity of 1.054 g/ml plus or minus 0.050 g/ml.

8. The formulation of claim 7 wherein the viscosity is between 5 cP and 20 cP.

9. A formulation comprising (w/w):
20% to 35% ammonio methacrylate copolymer type B USP/NF;
1% to 10% triethyl citrate; and
60% to 70% water.

10. The formulation of claim 9 wherein the ammonio methacrylate copolymer is EUDRAGIT RS 30 D brand polymethylmethacrylate.

11. A formulation comprising (w/w) of:
28% EUDRAGIT RS 30 D polymethylmethacrylate;
6% triethyl citrate; and
66% water.

12. A method for protecting pharmacologically-active substances applied in a temporary coating to a surface of a tooth comprising:
applying a pharmacologically inert barrier coating of a polymethylmethacrylate and a plasticizer on top of the temporary coating containing the pharmacologically-active substance to serve as a temporary mechanical barrier against the washings of saliva and abrasion caused by eating food.

13. The method of claim 12 wherein the pharmacologically-active substance(s) comprise one or more active agents of the type known to reduce caries when applied to the tooth.

14. The method of claim 13 wherein the pharmacologically-active substances are selected from the group consisting of chlorhexidine and fluoride.

15. The method of claim 12 wherein the polymethylmethacrylate is water-dispersed and the plasticizer is a pharmaceutical grade plasticizer selected from the group consisting of triethyl citrate, dibutyl sebacate, dibutyl phthalate, and diethyl phthalate.

16. The method of claim 12 wherein the polymethylmethacrylate is an ammonio methacrylate copolymer, type B USP/NF.

17. The method of claim 16 wherein the ammonio methacrylate copolymer is EUDRAGIT RS 30 D brand polymethylmethacrylate.

18. A method of preventing or reducing the incidence of caries in teeth, comprising the steps of:
a. applying a liquid coating of pharmacologically-active substances of the type used to reduce caries to a tooth surface; and
b. applying a pharmacologically inert barrier coating of a polymethylmethacrylate and a plasticizer on top of the coating containing the pharmacologically-active substance to serve as a temporary mechanical barrier against the washings of saliva and the abrasion from eating foods.
